(19) United States
(12) Patent Application Publication
(10) Pub. No.: US 2007/0282271 A1
(43) Pub. Date: Dec. 6, 2007

(54) Ostomy and percutaneous catheter protective and affixation device

(76) Inventors: Michael D. Kaplan, Franklin Lakes, NJ (US); Cornelius McCarthy, Jersey City, NJ (US); Steven M. Kaplan, New York, NY (US)

Correspondence Address: PEARL COHEN ZEDEK LATZER, LLP, 1500 BROADWAY 12TH FLOOR, NEW YORK, NY, 10036, US

(21) Appl. No.: 11/443,644

(22) Filed: May 31, 2006

(51) Int. Cl. A61M 5/32; A61M 31/00; A61M 37/00

(52) U.S. Cl. 604/174; 604/288.01

(57) ABSTRACT

The device protects the proximal extension of an indwelling catheter and its percutaneous puncture site from moisture, fluids and risk of infection and maintains a clean environment and a fluid-impermeable and bacteria growth-inhibiting seal against the patient's skin when bathing, showering or swimming, and also protects others from being exposed to the patient's blood and catheter at that time. A faceplate is adhered to the skin about the catheter exit site with medical adhesive, the proximal end of the catheter is secured to the faceplate to prevent it from moving from within the patient's body, and a water-impermeable housing is sealed to the faceplate, with the proximal end of the catheter extending into a cavity of the housing. An antimicrobial material may be disposed on the backside of the faceplate. The device allows a patient to shower, bathe and swim while preventing the catheter from being dislodged from the patient.

### BACKGROUND OF THE INVENTION

The invention relates to an indwelling percutaneous catheter. It specifically relates to protective guards and sleeves that maintain a clean and dry environment at the site where the percutaneous catheter exits the skin, while preventing movement of the catheter relative to the exit site.

Percutaneous catheters are flexible tubes used on a daily basis in medical practice for parenteral feeding or drainage, and many are long term, chronic catheters that are left in place within a patient for long periods of time. A catheter may be left in place, for example, to permit periodic blood transfusions, to perform renal dialysis, or to permit the periodic administration of antibiotics, chemotherapy treatment or parenteral nutrition. Such a catheter typically has its distal end positioned within the venous system of the patient or in other body locations including, but not limited to, the biliary, urinary system or peritoneal cavity. The proximal end of the catheter is located outside the patient's body and is periodically connected to medical equipment for, e.g., transfer of fluids or nutrition, transfusion, dialysis, etc. In the case of a biliary or urinary catheter, the catheter may be continuously or intermittently connected to a drainage bag.

Because indwelling, percutaneous catheters have the disadvantage of extending through an ostomy in the patient's skin, the exit site can serve as a portal for infectious agents, such as bacteria, fungi and viruses to enter into the system, and is thus prone to infection. Patients that use this type of catheter must, therefore, take extraordinary precautions to keep the proximal end of the catheter and the region surrounding puncture site in a clean and dry condition. Accordingly, such patients must avoid showering and swimming while indwelling catheters are in place. Naturally, this leads to a considerable discomfort on the part of the patient. In addition, exposure of the external portion of a catheter may be hazardous to others in a public facility such as a swimming pool, for example from blood or purulent infected material.

In addition, such indwelling, percutaneous catheters that extend through an ostomy in the patient's skin are typically not mounted or anchored to the outside of the patient in any way. As a result, even through normal daily movement, and certainly from more active movement, these catheters can be pulled from or pushed into the patient, thus dislodging the distal end of the catheter from its desired place within the patient's body. Such an occurrence can have disastrous consequences, in particular if not quickly detected by the patient or a physician.

Initially, patients or doctors protected these catheters from moisture by taping over them well with water-impermeable bandages prior to showering by the patient. However, this is a time consuming, uncomfortable and messy process that was not always successful and moreover did not allow for the patient to swim or bathe in a tub or shower. It also does not protect against exposure to sweat, which often leads to infection.

Alternatively, the catheters could be protected by a shield or drape that extends over a portion of the patient's body and covers the catheter and its entry site. For example, as shown in U.S. Pat. No. 6,571,395 (Korkor), catheters could be protected by a protective guard in the form of a water impermeable collar and shield that drapes around the patient's neck and over his shoulders, back and chest to brace the catheter against the user and to protect the catheter and its incision site while bathing. However, this excessively bulky shield is a one time use device and lends itself only to internal jugular vein and subclavian vein catheters. In addition, this shield does not allow for swimming or tub bathing, does not allow the patient's entire body to be cleaned and does not include any form of microbial growth prevention.

A commercial device distributed by SDA Product Inc., of North Miami Beach, Fla., known as the CD-1000 Adult Composite Dressing, is a dressing that is draped over the user's shoulder and the wound area to encompass the external catheter. The dressing has an aperture through its backing and adheres to skin using medical grade adhesive on the backing around the aperture, and has a transparent front for easy viewing of the wound and catheter. Similarly, U.S. Pat. No. 5,415,642 (Shepard) describes a smaller, fluid-impermeable protective shield that is adhered to the skin surrounding an ostomy using medical grade adhesive and provides a pouch-like cover extending from the adhered portion to protect the ostomy and the proximal end of an indwelling, percutaneous catheter from contact with water during showering, bathing or swimming. However, these catheter covers require some degree of expertise in applying the adhesive and must be repeated prior to every bathing event, which could be time consuming and frustrating for the patient. Additionally the daily or frequent application of the adhesive to the patient's skin could be irritating to the skin.

Other devices, such as those described in U.S. Pat. No. 4,213,458 (Nolan et al.), U.S. Pat. No. 4,419,100 (Alexander) and U.S. Pat. No. 4,889,534 (Mohiuddin et al.), provide another type of sealing attachment for an ostomy cover or a medical drainage pouch that could be used to enclose a percutaneous catheter. In these devices, an adhesive faceplate with an opening therethrough is adhered to the patient's skin about the ostomy, and a medical drainage pouch may be coupled to the ostomy faceplate using a coupling ring assembly. The coupling ring assembly may comprise collars on the faceplate and the drainage pouch that mate, either directly to each other or indirectly via a separate sealing ring that detachably connects the pouch and faceplate together. While these devices eliminate the necessity of daily or frequent application of the adhesive to the patient's skin, the adhesive that attaches that ostomy cover or faceplate to the patient's skin will necessarily become soaked with water or bodily fluid during showering or bathing. The wet adhesive surrounding the ostomy can easily promote bacterial growth and, as a result, infection, which is an especially undesirable occurrence close to an open wound.

Furthermore, in all of these devices, the proximal end of the indwelling, percutaneous catheter is left loosely hanging from the patient's body in an unsecure state. Such an unsecured indwelling, percutaneous catheter can easily be pulled outward or pushed inward, thereby dislodging its distal end from the desired location within the patient's body or causing the proximal end of the catheter to slide into the patient's body. This would be especially problematic if one of the Nolan et al., Alexander and Mohiuddin et al. devices are used, because the drainage pouches allow the catheter greater freedom of movement, thereby creating a greater risk of the catheter being dislodged from its desired place within the patient. None of the prior art devices provides any way for securing the percutaneous catheter to the outside of the patient, while also providing an infection resistant moisture barrier to enable the patient to swim and/or bathe without fear of infection at that site.

### SUMMARY OF THE INVENTION

It is an object of the present invention to provide a device that provides a fluid-impermeable seal against the patient's skin to allow the catheter exit site to be protected from moisture and fluids.

It is also an object of the present invention to provide such a device that prevents bacterial or other microbial growth and infection of the patient's skin at the site of the fluid-impermeable seal about the catheter exit site.

It is a further object of the present invention to provide such a device that also prevents the catheter from being moved in or out of the patient or from being moved laterally at the exit site.

The objects and advantages of the invention are accomplished by providing an ostomy and percutaneous catheter protective and affixation device in the form of a protective shield around the proximal extension of an indwelling catheter and its percutaneous puncture site for maintaining a clean environment and a fluid-impermeable and bacteria growth-inhibiting seal against the patient's skin to allow the catheter exit site to be protected from moisture, fluids and risk of infection. This device also affixes the proximal end of the percutaneous catheter to the patient's skin or to the protective shield in order to secure the proximal end of the indwelling percutaneous catheter.

In accordance with a preferred embodiment of the invention, the protective shield comprises a fluid-impermeable housing that has an open end and a closed end, with a mouth at its open end and a sealing ring surrounding the mouth. The housing is preferably composed of a flexible material that is substantially impervious to infectious agents and has a sufficient interior volume to receive the proximal extension of the indwelling catheter.

The protective shield also comprises a base or faceplate, preferably composed of a flexible material that is substantially impervious to infectious agents, that is affixed to the skin of the patient around the catheter exit site. The base comprises an opening through which the proximal end of the percutaneous catheter exits, and which opening either is wide enough to accommodate a bulky catheter end or has radial slits about the edge of the opening to allow a bulky catheter end to fit through the opening when the device is being changed. The opening is surrounded by a sealing ring that mates with the sealing ring surrounding the mouth of the protective shield to form a watertight seal. A fluid impermeable medical grade adhesive is disposed on the back surface of the base, allowing the protective shield to be attached to the skin that surrounds the catheter exit site. It is preferable that an antibacterial and antimicrobial material also be disposed upon the back surface of the base about the opening in order, such as a silver nitrate impregnated material, in order to impede and prevent bacterial growth.

The upper surface of the adhering region of the faceplate contains a fastening structure to secure the proximal end of the percutaneous catheter to the base of the protective shield. The fastening device can include suitable clamps or clips, such as the StatLock or any other similar device, wherein the catheter is secured with preferably a low profile.

In operation, the faceplate is first placed onto the patient by threading the proximal end of the percutaneous catheter through the opening of the faceplate, and a fluid impermeable medical grade adhesive is used to affix the faceplate to the patient about the catheter exit site. Next, the proximal end of the catheter is secured to the faceplate using the fastening device. The loose-hanging proximal end of the catheter can be inserted through the housing mouth and the protective housing can then be slipped over the proximal end of the catheter, and the housing is sealed against the faceplate by mating the sealing ring of the protective housing mating with the sealing ring of the faceplate opening.

The faceplate can then remain in place and be changed as infrequently as every few weeks or as often needed, by a home health nurse or dialysis nurse. The protective cover will be supplied in varying sizes depending on the length of the peripheral portion of the patient's catheter. A new sterile, water impermeable cover will be applied by the patient himself, a family member, a home health nurse or a dialysis nurse, or any other qualified individual, prior to the patient showering, bathing or swimming. Following the bathing activity, the bag will be discarded, and a “breathable”, sterile cover can be applied until the next swimming/bathing event or until the next dialysis session.

A significant feature of the current invention is the fact that the faceplate may be used multiple times before the need for a new device. It is also unique in that it provides for a fluid impermeable and infection resistant environment while at the same time allowing for the catheter to be secured while the patient is bathing. The covers will be interchangeable between a water-proof cover while swimming, showering and bathing, and a “breathable” gauze (or substitute) cover at other times.

A significant feature of the present invention is that the protective shield constructed in accordance with the invention provides a fluid-impermeable and infection resistant seal when applied to a patient, while at the same time ensuring that the catheter is not dislodged from its position within the patient.

Also provided in accordance with the present invention is a method for inhibiting contamination of an indwelling catheter entry site on a patient while preventing dislodging of the indwelling catheter. The method involves the steps of identifying the entry site of the catheter, advancing the proximal end of the catheter through the opening in the faceplate, adhering the faceplate to the skin of the patient around the catheter exit site, securing the catheter to the faceplate, inserting the proximal end of the catheter into the cavity of the housing, and sealing the mouth of the housing to the faceplate.

### BRIEF DESCRIPTION OF THE DRAWINGS

The above and other objects and advantages of the invention will be apparent upon consideration of the following detailed description, taken in conjunction with the accompanying drawings, in which the reference characters refer to like parts throughout and in which:

FIG. 1 is a perspective view of a preferred embodiment of the ostomy and percutaneous catheter protective and affixation device showing the faceplate to be adhered to the skin of a patient and the housing to be attached to the faceplate;

FIG. 2 is a side cross-sectional view of the device prior to attachment of the housing to the faceplate;

FIG. 3 is a partial side cross-sectional view of an alternate embodiment of the faceplate;

FIG. 4 is side cross-sectional view of the device showing the faceplate adhered to the skin of a patient about the catheter and the housing attached to the faceplate.

### DETAILED DESCRIPTION OF THE INVENTION

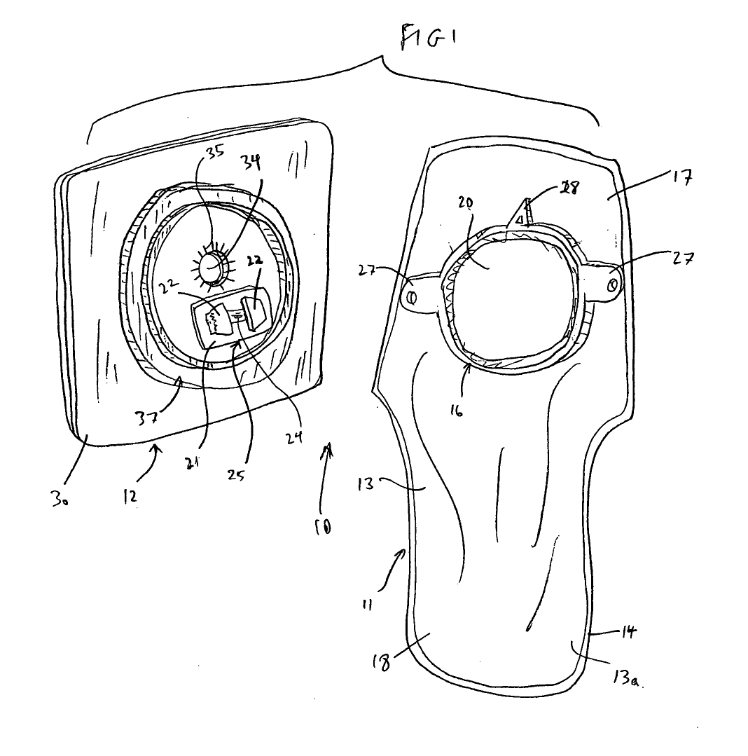

FIG. 1 illustrates a two-part ostomy and percutaneous catheter protective and affixation device 10 consisting of a housing assembly 11 and a faceplate assembly 12. The housing assembly 11 for enclosing the proximal end of the catheter is preferably in the form of a pouch or bag, having a flexible peripheral wall 13. Because housing 11 will be exposed to the open ostomy, wall 13 should be preferably composed of a fluid-impermeable material that is substantially impervious to bacterium, fungi, viruses and other infectious agents. Various materials such as latex and silicone rubber are available for this purpose, as will be appreciated by one skilled in the art. The housing may also be formed of two panels 13a and 13b joined together by a peripheral zone of heat sealing 14. The housing wall 13 defines an internal cavity 19 that has a sufficient interior volume to receive the proximal extension of the indwelling catheter.

In a preferred embodiment, wall 13 of housing assembly 11 has an open upper end 17 and a closed lower end 18. The open end 17 allows a catheter to be received into a closed chamber formed by the inner surface of the wall 13. In certain embodiments, it may be desirable for housing 11 to terminate in an opening at the lower end 18 of wall 13. Where such an opening is provided, a suitable clamp, such as the clamp disclosed in U.S. Pat. No. 3,523,534 (Nolan), can be used to maintain the opening at the lower end 18 in a closed condition.

A mouth 20 is disposed through the upper end 17 of wall 13 of housing 11, which aperture 20 is bordered by a first sealing ring 16, which is secured to wall 13 (or one wall panel 13a) of housing 11 by heat sealing or by any other suitable means. Sealing ring 16 preferably has a generally circular configuration, defining a central opening that communicates with the interior 19 of housing 11 through mouth 20 in the upper portion of wall 13. A pair of apertured tongues 27 may project laterally from opposite sides of the sealing ring 16 for the attachment of a suitable support belt, if the use of such a belt is desired by the wearer. An integral tab 28 may also project radially outwardly from the periphery of sealing ring 16 to serve as a handle for pulling sealing ring 16 away from the faceplate assembly 12 during an uncoupling operation.

In a preferred embodiment, faceplate 12 assembly includes a base 30 that should be highly flexible, so that it will conform readily to body contours and body movements, and be relatively strong and durable. In a preferred embodiment, base 30 is formed of a gas-penetrable but water resistant microporous material, for relatively long-term adhesion to the skin. For example, a reinforced non-woven cellulosic material of the type sold under the Kaycel trademark by Kimberly-Clark Corporation, Neenah, Wis., which is not only air-pervious but is surface-coated with an ethyl vinyl acetate latex emulsion so that it is also heat-sealable, may be used. A porous, expanded, high-density polyethylene or polypropylene film of the type marketed under the designation Delnet by Hercules Incorporated, Wilmington, Del. may be included for strength and soil resistance, and other porous thermoplastic films or membranes such as Gore-tex, a microporous polytetrafluoroethylene membrane marketed by W. L. Gore & Associates, Newark, Del., may also be employed. Similarly, it is possible to use copolymer films of ethylene and vinyl acetate laminated to non-woven polyester or non-woven rayon layers. Alternatively, base 30 may comprise one or more additional support layers to produce a laminate or sandwich configuration for imparting increased structural integrity.

The base 30 may have any outline shape, such as circular, oval, square, or generally rectangular as shown in FIG. 1, and is provided with a small central opening 34, as shown most clearly in FIG. 1, through which the percutaneous catheter is threaded. Opening 34 can be made wide enough to accommodate even the most bulky catheter end. Alternatively, in order to maintain a tight fit around the shaft of the catheter proximal end, opening 34 is preferably provided with radial slits 35 radiating outward from the edge of the opening to allow a bulky catheter end to fit through the opening when the faceplate assembly 12 is being changed (after the catheter is in place). As a result of slits 35, opening 34 expands to allow the bulky catheter end to pass through, after which opening 34 returns to its smaller size for attachment of the faceplate 12 to the patient's skin.

A second sealing ring 37 is mounted upon base 30 about opening 34 and is secured to base 30 of faceplate 12 by heat sealing or by any other suitable means. Second sealing ring 37 is adapted to mate with first sealing ring 16 to produce a highly effective watertight sealing interlock between the parts, and for this the sealing rings 16 and 37 may have any one of many suitable configurations that are well known in the art. It is to be understood that many different types or structures of sealing or coupling ring assemblies may be used, such as any of those taught in U.S. Pat. Nos. 4,419,100, 4,786,285 and 4,889,534 assigned to Hollister Incorporated of Libertyville, Ill., and U.S. Pat. No. 5,257,981, all of which are incorporated herein by reference, as well as others.

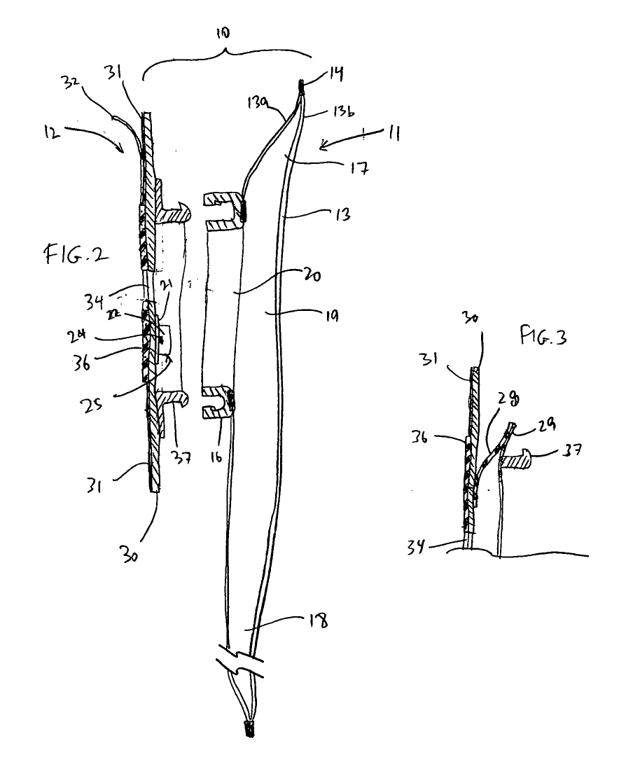

As shown in FIG. 2, the back or rear surface of base 30 is provided with an adhesive 31 suitable for application to the skin, such as by being coated with a medical-grade pressure-sensitive adhesive 31. The adhesive 31 is preferably fluid impermeable and is preferably applied around the entire back surface of the base 30 without discontinuity so that a fluid impermeable seal can be formed when the base 30 is applied to the patient's skin. The adhesive coating 31 may be applied to the back surface of base 30 in accordance with any of a variety of conventional techniques.

It is preferable that, prior to use, the adhesive 31 on the back surface of base 30 be covered by a release layer 32, such as backing sheets. Upon removal of the release layer 32, the faceplate 12 may be adhesively secured to the patient's skin in the peristomal region. Release layer 32 can be adapted so as to be easily removable after the proximal end of a catheter has been inserted through opening 34, so that the catheter may be inserted through opening 34 without sticking to the adhesive 31. Other release layer configurations can also readily be devised by one of skill in the art.

In a preferred embodiment, faceplate 12 contains, in addition to its fluid impervious composition, some infection resistant, antibacterial and/or antimicrobial treatment in order to ensure that the peristomal region remains free of potential infectious agents and contamination. In one embodiment, the region about opening 34 on the back side of the faceplate 12 can contain, bear or be impregnated with such a material 36. One preferred material is a silver nitrate plated polymeric substrate, such as described in U.S. Pat. Nos. 6,087,549 and 7,005,556, both assigned to Argentum Medical LLC of Willowbrook, Ill., which are incorporated herein by reference. Preferred materials are Silverlon® by Argentum Medical LLC and Acticoat® by Smith & Nephew plc of London, England, which provide a controlled release of silver ions to protect the wound and dressing from bacterial contamination. Adhesive 31 may be applied to the back surface of the antimicrobial material 36 as well to adhere it to the patient's skin. Alternatively, adhesive 31 may be applied around the antimicrobial material 36 on the back surface of base 30 so as to allow the antimicrobial material 36 to have greater contact with the patient's skin.

In addition, it is apparent that the use of protective covers to enclose percutaneous catheters having relatively long proximal extensions protruding from the skin can, upon normal movement of the patient, cause the catheter to move relative to the patient and cause the distal end of the catheter to be dislodged from its position within the patient. In order to minimize this, one or more structures are preferably provided to immobilize the proximal end of the catheter.

Accordingly, faceplate assembly 12 also includes a fastening structure 25 to secure the proximal end of the percutaneous catheter so that it does not move relative to the patient. In a preferred embodiment, such a fastening structure is incorporated on base 30, within the bounds of the sealing ring 37. In certain embodiments, the fastening structure 25 may be bonded directly to or mounted directly onto base 30, while in other embodiments the fastening structure 25 is mounted onto a base or pad 21 that is in turn bonded to or mounted onto base 30. Such a fastening device can be clamps, clips, brackets 22 or any one or combination of those known to the art that are generally used to secure devices, particularly catheters and their associated tubing to the patient. It is also preferred that such a fastening structure 25 have a low profile and most preferably not protrude outward beyond the height of sealing ring 37 in order that it not interfere with the mating of the sealing rings 16,37 or tend to cause tears in wall 13 of housing 11 due to its protrusion.

Many types of catheter fastening structures may be used, such as those shown in U.S. Pat. Nos. 5,722,959, 5,855,591, 6,117,163 and 7,014,627 to Bierman, all of which are incorporated herein by reference, as well as others assigned to Venetec International, Inc. of San Diego, Calif. Preferred fastening devices are made by Venetec International under the name StatLock®, and one example of a preferred embodiment of this brand is known as the PICC Plus/PICC model (used for peripherally inserted central catheters). In a typical fastening structure 25, the brackets 22 are closed around the proximal end of the catheter as it exits through opening 34 in base 30 to thereby against the catheter against base 30. In certain embodiments, the spacing of the brackets 22 is adjustable to accommodate catheters of different widths, for example via movable posts 24 upon which the brackets 22 are mounted. To use this embodiment of the fastening structure 25, the brackets 22 are opened, the moveable posts 24 are adjusted to fit the particular catheter being used, the catheter is placed between the brackets 22, and the brackets 22 are closed around the proximal end of the catheter to secure the catheter against base 30 of faceplate assembly 12 and to prevent it from moving.

To use the ostomy and percutaneous catheter protective and affixation device 10, the proximal end of the indwelling catheter is threaded through opening 34 in faceplate assembly 12, from the back side of base 30 to the front of base 30, expanding slits 35 in the process, if necessary. Release layer 32 is then removed from the back surface of the base 30 to expose the adhesive 31. The base 26 is then applied and adhered to the skin of the patient such that the opening 34 is directly above the catheter exit site 12. Then, the proximal end of the catheter is secured to faceplate 12 using fastening structure 25 so as to be immobilized relative to the catheter exit site and opening 34.

Once faceplate assembly 12 is applied to the patient's skin at the catheter exit site, housing assembly 11 must be attached to faceplate 12 so as to retain the proximal end of the catheter and also sealingly engage against faceplate 12. It is contemplated that the housing 11 as described can be sold in individually wrapped, sterile packages that can be opened immediately prior to use. First, the portion of the proximal end of the catheter that is not bound using fastening structure 25 is inserted into the cavity 19 of housing assembly 11 through mouth 20. Housing 11 is then attached to faceplate assembly 12 by pressing first sealing ring 16 on wall 13 of housing 11 against second sealing ring 37 on base 30 of faceplate assembly 12 so that their structures mate, as discussed above, to provide a fluid-impermeable seal between them. The catheter cover 10 thereby allows the patient to swim, bathe, etc. without undue risk of infection. The catheter cover 10 also protects public and private swimming and bathing facilities from exposure to blood and other potentially hazardous fluids that may be present on the outside of the catheter. FIG. 4 shows a cross-sectional view of the device with the faceplate 12 adhered to the skin of a patient about the percutaneous catheter exit site, with the housing 11 attached to the faceplate 12 and the proximal end of the catheter disposed within cavity 19 of housing 11.

In order to remove housing assembly 11 from faceplate assembly 12, sealing ring 16 on wall 13 of housing 11 must be uncoupled from sealing ring 37 on base 30. This is achieved by gripping tab 28 on sealing ring 16 and pulling it radially outwardly to disengage sealing ring 16 on the housing assembly 11 from sealing ring 37 on faceplate assembly 12. During such operation, the patient (or other person) immobilizes sealing ring 37 by gripping base 30 in the area adjacent tab 28. The housing 11 is then moved downward to slide it off the proximal end of the catheter and discarded. It is contemplated that the housing 11 can be sold as a relatively inexpensive, one-time-use disposable product. Then, if desired, fastening structure 25 is opened, releasing the catheter for use or manipulation by a physician.

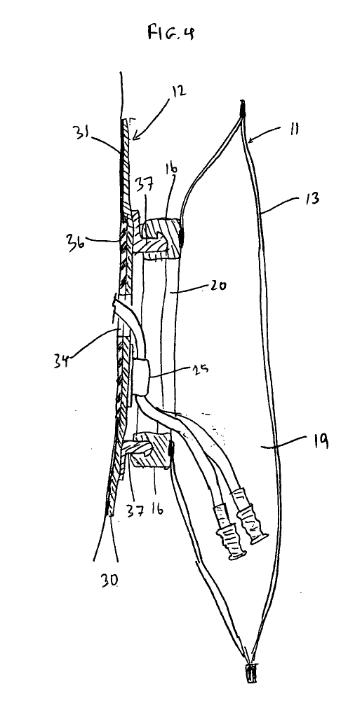

In a first embodiment, as shown in FIG. 2, base 30 is a flat pad that is adhered directly to the patient's skin. But, because the pulling action during uncoupling of the sealing rings may transmit some pulling forces to the faceplate assembly 12, it may also cause some patient discomfort or result in separation (or weakening) of the adhesive seal between the patient and the adhesive 31 on faceplate assembly 12. Accordingly, in an alternative embodiment, it may be desirable not to form base 30 as a flat pad that is adhered directly to the patient's skin but rather to incorporate within the structure of the faceplate assembly 12 any one of the flexible webs, accordion flanges or extra faceplate assembly portions that are known in the art to lessen or eliminate such forces. Structures that resist or minimize such pulling forces are shown, for example, in U.S. Pat. Nos. 4,419,100 and 4,889,534 assigned to Hollister Incorporated of Libertyville, Ill., and U.S. Pat. Nos. 4,648,875, 4,664,661, 4,685,990 and 4,846,820 assigned to E. R. Squibb & Sons, Inc. of Princeton, N.J., all of which are incorporated herein by reference, as well as others. For example, FIG. 3 shows an alternate embodiment of the faceplate 12, wherein base 30 is connected a flexible annular web 28 that is in turn connected to sealing ring 37 by way of flange 29, such as described in U.S. Pat. No. 4,419,100. This structure, wherein the inner edge of annular web 28 is connected to the front side of base 30 about aperture 34 and the outer edge of annular web 28 is connected to sealing ring 37 via flange 29, allows only limited movement of sealing ring 37 in generally axial directions normal to base 30, thereby providing relief from the pushing or pulling forces that would be applied to faceplate 12 and cause discomfort to the patient during attachment or removal of housing 11.

It should be understood that a catheter cover in accordance with the present invention can be used with virtually any type of device that passes through the skin, such as to permit the passage of fluids, electrical currents, and the like. Thus, the terms “catheter”, “percutaneous catheter” and “indwelling catheter” as used herein to describe the present invention are intended to include all devices that meet this description.

Although this invention has been described in terms of certain preferred embodiments, other embodiments that are apparent to those of ordinary skill in the art are also within the scope of this invention. Accordingly, the scope of the present invention is intended to be defined only by reference to the appended claims.

What is claimed is:

1. A device for protecting a percutaneous catheter, comprising:
a flexible faceplate adapted to be adhesively secured to a peristomal surface, said faceplate having
a front side and a back side,
an aperture,
a first sealing ring mounted on said front side about said aperture, and
a catheter securing means mounted on said front side for gripping the proximal end of said percutaneous catheter after it is passed through said aperture;
an adhesive disposed on said back side of said faceplate and suitable for adhering said faceplate to the skin of a patient; and
a fluid-impermeable housing for receiving the proximal end of a catheter projecting through said aperture, said housing having an opening at one open end and a second sealing ring mounted on said housing about said opening, said second sealing ring adapted to detachably mate with said first sealing ring to form a fluid-impermeable seal.

2. The protective device according to claim 1, wherein said housing is substantially impervious to bacteria, fungi and viruses.

3. The protective device according to claim 1, wherein said faceplate comprises a flat annular flange.

4. The protective device according to claim 1, wherein said faceplate comprises a flexible annular web having a pair of concentric edge portions, one of said edge portions being connected to said first sealing ring and the other of said edge portions being connected to said front side of said faceplate about said aperture, said flexible web allowing limited movement of said first sealing ring in generally axial directions normal to said faceplate.

5. The protective device according to claim 1, wherein said faceplate further comprises on said back side thereof an antimicrobial material.

6. The protective device according to claim 5, wherein said antimicrobial material comprises silver.

7. The protective device according to claim 5, wherein said antimicrobial material is disposed between said adhesive and said aperture.

8. The protective device according to claim 1, wherein said catheter securing means comprises at least one clamp, clip or bracket to secure said catheter to the front side of said faceplate.

9. The protective device according to claim 1, wherein said faceplate further comprises a plurality of slits formed through said faceplate and extending radially from said aperture.

10. A method for inhibiting contamination of an indwelling catheter entry site on a patient while preventing dislodging of the indwelling catheter, comprising the steps of
identifying the entry site of the catheter, said catheter having a distal end inside the body of a patient and a proximal end projecting through the entry site and out of the body of the patient;
advancing the proximal end of the catheter through an aperture in a faceplate, said faceplate having a sealing ring about said opening,
adhering the faceplate to the skin of the patient such that said aperture is around the catheter exit site,
securing the proximal end of the catheter to the faceplate,
inserting the proximal end of the catheter through the opening of a housing and into a cavity of said housing, said housing having a sealing ring about said opening, and
mating the sealing ring about the opening of the housing to the sealing ring on the faceplate to form a seal.

11. The method of claim 10 wherein said step of securing the catheter to the faceplate comprises using at least one clamp, clip or bracket to secure the proximal end of the catheter to the faceplate.

12. The method of claim 10 further comprising the step of disposing on a back side of said faceplate an antimicrobial material.

13. The method of claim 12, wherein the step of adhering the faceplate comprises disposing adhesive on the back side of said faceplate, and the step of disposing on an antimicrobial material comprises said antimicrobial material between said adhesive and said aperture.

14. The method of claim 12, wherein said antimicrobial material comprises silver.

15. A device for maintaining the position of a percutaneous catheter relative to a patient, comprising:
a flexible faceplate adapted to be adhesively secured to a peristomal surface, said faceplate having a front side and a back side,
an aperture disposed through said faceplate,
a catheter securing means mounted on said front side of said faceplate for gripping a proximal end of said percutaneous catheter after it is passed through said aperture;
an adhesive disposed on said back side of said faceplate and suitable for adhering said faceplate to the skin of a patient.

16. The device according to claim 15, further comprising a first sealing ring mounted on said front side about said aperture and a fluid-impermeable housing for receiving the proximal end of a catheter projecting through said aperture, said housing having an opening at one open end and a second sealing ring mounted on said housing about said opening, said second sealing ring adapted to detachably mate with said first sealing ring to form a fluid-impermeable seal.

17. The device according to claim 15, wherein said catheter securing means comprises at least one clamp, clip or bracket to secure said catheter to the front side of said faceplate.

18. The device according to claim 15, wherein said faceplate comprises a flat annular flange.

19. The device according to claim 15, wherein said faceplate comprises a flexible annular web having a pair of concentric edge portions, one of said edge portions being connected to said first sealing ring and the other of said edge portions being connected to said front side of said faceplate about said aperture, said flexible web allowing limited movement of said first sealing ring in generally axial directions normal to said faceplate.

20. The device according to claim 15, further comprising an antimicrobial material on said back side of said faceplate.

21. The device according to claim 20, wherein said antimicrobial material comprises silver.

22. The device according to claim 20, wherein said antimicrobial material is disposed between said adhesive and said aperture.

23. The device according to claim 15, further comprising a plurality of slits formed through said faceplate and radially extending from the edge of said aperture.
